(19) United States
(12) Patent Application Publication
(10) Pub. No.: US 2009/0306701 A1
(43) Pub. Date: Dec. 10, 2009

(54) Vascular access sheath with integrated return electrode

(75) Inventor: Stephen C. Porter, Oakland, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 12/387,272

(22) Filed: Apr. 30, 2009

Related U.S. Application Data
(60) Provisional application No. 61/131,578, filed on Jun. 10, 2008.

(51) Int. Cl. A61M 29/02; A61M 25/01

(52) U.S. Cl. 606/195; 604/523; 604/21

(57) ABSTRACT

Described herein are vascular access sheaths having an integrated electrode therein. Also described are assemblies comprising the sheaths as well as methods of making and using the vascular access sheaths.

### CROSS-REFERENCE TO RELATED APPLICATIONS

This application claims the benefit of U.S. provisional patent application No. 61/131,578, filed Jun. 10, 2008, the disclosure of which is incorporated by reference in its entirety for all purposes.

### TECHNICAL FIELD

This disclosure relates to vascular access sheaths for providing an access site for interventional devices to the vasculature. In particular, disclosed are vascular access sheaths with integrated electrodes for facilitating the passage of electrical signals to or from the body. More particularly, the sheaths may be used to facilitate the electrolytic separation of an implant, such as an embolic device, from a delivery member.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel.

One widely used vaso-occlusive device is a helical wire coil having windings that may be dimensioned to engage the walls of the vessels. (See, e.g., U.S. Pat. No. 4,994,069 to Ritchart et al.). Variations of such devices include polymeric coatings or attached polymeric filaments have also been described. See, e.g., U.S. Pat. Nos. 5,226,911; 5,935,145; 6,033,423; 6,280,457; 6,287,318; 6,299,627; 5,582,619; 5,833,705; 5,853,418; 6,004,338; 6,013,084; 6,179,857; and 6,193,728.

Typically, implantable devices include a detachment mechanism in order to be released from the deployment mechanism (e.g., attached wire). One detachment mechanism involves the use of electrolytic means to detach the vaso-occlusive member from the pusher. See, e.g., U.S. Pat. Nos. 5,122,136; 5,354,295; 6,620,152; 6,425,893; and 5,976,131, all to Guglielmi et al. and U.S. Pat. No. 6,623,493, describing a vaso-occlusive assembly with multiple electrolytic detaching points. U.S. Pat. Nos. 6,589,236 and 6,409,721 describe assemblies containing an electrolytically severable joint. The coil is bonded via a metal-to-metal joint to the distal end of the pusher. The pusher and coil are made of dissimilar metals. The coil-carrying pusher is advanced through the catheter to the site and a small electrical current is passed through the pusher-coil assembly. The current causes the joint between the pusher and the coil to be severed via electrolysis. The pusher may then be retracted leaving the detached coil at an exact position within the vessel. Since no significant mechanical force is applied to the coil during electrolytic detachment, highly accurate coil placement is readily achieved. In addition, the electric current may facilitate thrombus formation at the coil site.

The circuit involved in the electrolytic coil detachment arrangements generally includes a power source having its positive terminal coupled to the sacrificial link via a guidewire, for example. More specifically, a positive electric current of approximately 0.01 to 2 milliamps is applied to the guidewire which is coupled to the sacrificial link that is intended to undergo electrolytic disintegration and which initially couples the implant (e.g., the vaso-occlusion device) to the guidewire. The negative terminal of the power source is typically coupled to an electrode that is placed over and in contact with the patient's skin. This can be accomplished, for example, with a large skin electrode such as a ground pad or needle. Thus, the current path flowing from the detachment zone (etch site on the sacrificial link) to the ground electrode includes the patient's fluids and tissues. While the impedance to current flow is generally predictable through the delivery member or guidewire (approximately 200 ohms), the additional impedance of the current's return path to the ground electrode is dependent on variable impedances which depend on the blood milieu of the aneurysm, density of coil packing in the aneurysm, conductivity of the aneurysm wall, patient's tissue and return skin electrode, and fluid volume, for example. Large unanticipated patient-to-patient variations in the impedance of the electrical connection can be problematic when a high degree of precision in delivering specific current loads, or measuring accurate voltages is required. In the case of electrolytic coil detachment these variations can manifest in poor or false detection of implant detachment, variation of current control system performance, and/or increased duration of the detachment process.

Another return electrode or cathode arrangement is disclosed in U.S. Pat. No. 5,354,295 to Guglielmi et al. In that arrangement, the microcatheter is supplied with an end electrode. More specifically, the electrode extends distally from the microcatheter and is coupled to an electrical conductor or wire disposed along the length of the microcatheter. The wire is ultimately led back to the negative terminal of the power source so that the electrode (ring electrode) is used as the cathode during electrothrombosis instead of an exterior skin electrode.

U.S. Pat. No. 6,059,779 discloses a catheter for introducing a delivery member such as a guidewire having an implant coupled thereto via an electrolytically disintegratable link that is coupled to the anode of a power supply having an anode and a cathode. The catheter comprises an elongated tubular member having an outer surface, proximal and distal end portions, a lumen extending between those portions, an electrode and a conductor. The electrode is disposed within the outer surface of the tubular member in the vicinity of the distal end portion and has a surface that faces and communicates with the lumen. The electrode is electrically coupled to the conductor which, in turn, is adapted for coupling to the power supply cathode such that when the electrolytically disintegratable link is placed in an ionic solution, such as can be found in an aneurysm, and positive current is provided from the power supply to the link, current from the link may return to the cathode of the power supply via the electrode.

However, there remains need for devices and assemblies for delivering electrolytically detachable implantable devices that do not require (1) a needle stick for grounding and/or (2) complex return electrode configurations that extend into the vasculature to the site of implantation.

### SUMMARY

Described herein are vascular access sheaths that function as electrodes for the passage of electrical signals to or from the body in situations where large unanticipated patient-to-patient variations in the impedance of an electrical connection can be problematic. In particular, they may serve as grounding electrodes for any electrolytically detachable device. Methods of using these sheaths to deliver implants to the vasculature of a patient are also described.

In one aspect, described herein is a vascular access sheath comprising a tubular element having an inner and outer surface and an electrically conductive element on the inner and/or outer surface of the tubular element. In certain embodiments, the electrically conductive element is on the outer surface of the tubular element, for example, a layer of conductive material that at least partially covers the tubular element.

In any of the devices described herein, the conductive layer may comprise a metal and/or polymer. For example, the devices may include a metal selected from the group of metals or alloys comprising at least one of gold, platinum, silver, tantalum, palladium, tungsten, titanium, iron, cobalt, chromium, nickel, aluminum, copper. In certain embodiments, the conductive layer comprises titanium-nitride, graphite. In other embodiments, the conductive layer comprises a polymer.

Furthermore, in any of the devices described herein, the electrically conductive element may comprise one or more conductive elements selected from the group consisting of coatings, coils, braids, bands and wires.

Any of the devices described herein may further comprise a hydrophilic coating.

In another aspect, described herein is an assembly comprising: any of the vascular access sheaths described herein; a delivery catheter; and an electrolytically detachable implantable device. In certain embodiments, the electrolytically detachable implantable device comprises a vaso-occlusive device, for example, a vaso-occlusive coil.

In yet another aspect, described herein is a method of delivering or receiving an electrical signal from a patient body, the method comprising: introducing, into the vasculature of a subject, a vasculature access sheath as described herein; and attaching the sheath to an electrical circuit. In certain embodiments, the electrical circuit comprises an electrolytically detachable implant (e.g., vaso-occlusive device such as a vaso-occlusive coil), for example, an electrolytically detachable implant that is deployed using a catheter that extends into the vasculature through the vasculature access sheath.

In a still further aspect, provided herein is a method of occluding a body cavity comprising; introducing, into the vasculature of a subject, a vasculature access sheath as described herein; and deploying an electrolytically detachable vaso-occlusive device into the vasculature of the patient through the vascular access sheath. In certain embodiments, the body cavity is an aneurysm.

These and other embodiments will readily occur to those of skill in the art in light of the disclosure herein.

### BRIEF DESCRIPTION OF THE FIGURES

FIG. 1 is an overview of an exemplary vascular sheath as described herein.

### DETAILED DESCRIPTION

Vascular access sheath designs for delivery of implants comprising electrolytic detachment junctions and assemblies comprising these catheter designs are described. The sheaths described herein can be utilized in vascular and neurovascular indications and are particularly useful in delivering embolic devices to aneurysms located within, for example, small-diameter, curved or otherwise difficult to access vasculature, such as cerebral aneurysms. Methods of making and using these sheath designs are also described.

Currently, the gold-standard method of delivering vaso-occlusive devices is via electrolytic detachment (e.g., GDC coils). While electrolytic detachment solves the drawbacks of earlier mechanical detachments (e.g., the need for the mechanism to be fully inside the catheter in order to remain engaged), electrolytic detachment still requires a return path for the current, which is typically accomplished by inserting a needle into the subject or by using complex catheter configurations having a return electrode path near the detachment junction.

Since all patients undergoing interventional procedures require a vascular access sheath to provide primary access to the vasculature, the sheaths described herein provide an electrical return path without the need for a needle puncture or complex catheter designs. Furthermore, since the sheath penetrates through the soft tissue and into the vasculature, the electrical resistance of the circuit may be more reproducible than a needle stick. In addition, the depth of penetration of the sheath provides a larger surface area as compared to a needle stick, which reduces current-induced tissue burning and overall circuit impedance.

Thus, described herein are vascular access sheaths that provide a current return pathway for electrolytically detachable implantable devices without the need for a needle stick or complex catheters designs with return electrodes extending through the delivery catheter to the detachment junction.

All publications, patents and patent applications cited herein, whether above or below, are hereby incorporated by reference in their entirety.

It must be noted that, as used in this specification and the appended claims, the singular forms “a”, “an”, and “the” include plural references unless the content clearly dictates otherwise.

The sheath designs described herein include a electrically conductive element on the external and/or internal surface of the tubular sheath body and an electrical coupling to allow for connection to the power supply (e.g., GDC power supply).

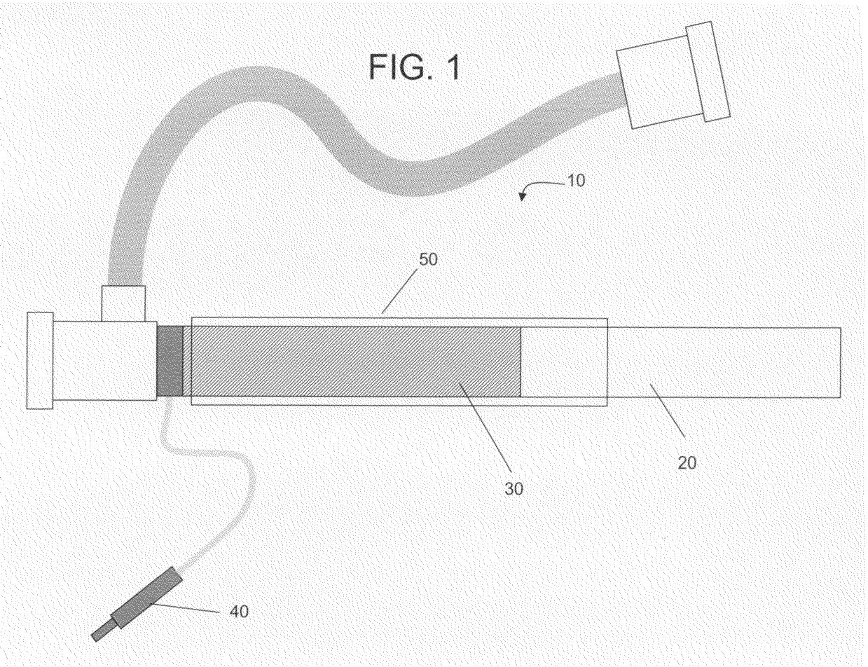

As shown in FIG. 1, in certain embodiments, the sheath 10 comprises a conductive layer 10 over part of the tubular body 20 of the sheath and an electrical coupling 40 to power supply. Also shown is optional hydrophilic coating 50. The optional hydrophilic coating can be any material that does not significantly reduce the circuit impedance and may reduce problems with hemocompatibility. Furthermore, although depicted in FIG. 1 as a continuous covering of a part of the sheath, it will be apparent the whole sheath can include a conductive layer and/or that the conductive layer may be discontinuous along the length of the sheath.

In embodiments in which the conductive material comprises a coating on the inner and/or outer surface of the sheath body, any conductive material can be used. Preferably, the conductive coating does not create a detrimental reduction reaction which may cause a biological response and does not cause localized thrombotic reaction. The coating may be adhered to the sheath body using any suitable means, including, but not limited to, dip coating, gluing, UV curing, soldering, and the like. Non-limiting examples of suitable conductive coating materials include metals (e.g., gold, platinum, stainless steel and other metal alloys), carbon (e.g., graphite) and/or conductive polymers.

In other embodiments, the return electrode (conductive element) comprises one or more conductive elements, for example coils, braids, bands and/or wires. The elements may be positioned anywhere on the sheath as long as they function as a return electrode.

The tubular body of the sheaths described herein may be made of any biologically compatible material(s), including for example, polyethylene, polypropylene, Nylon, polyurethane, polyimides, polyvinyl chloride, polysulfones, polyfluorocarbons, polyethylene terephthalate, their mixtures, copolymers; and/or polyester elastomers.

The vascular sheaths described herein can be adapted to be used with any vascularly implantable device that is detached by application of electrical energy, including but not limited to, vaso-occlusive devices, stents, and the like. In a preferred embodiment, the implant comprises a vaso-occlusive device, for example a vaso-occlusive device comprising a helical wire coil having windings that may be dimensioned to engage the walls of the vessels. (See, e.g., U.S. Pat. No. 4,994,069 to Ritchart et al.). Variations of such devices include polymeric coatings or attached polymeric filaments have also been described. See, e.g., U.S. Pat. Nos. 5,226,911; 5,935,145; 6,033,423; 6,280,457; 6,287,318; and 6,299,627. In addition, coil designs including stretch-resistant members that run through the lumen of the helical vaso-occlusive coil have also been described. See, e.g., U.S. Pat. Nos. 5,582,619; 5,833,705; 5,853,418; 6,004,338; 6,013,084; 6,179,857; and 6,193,728.

Suitable vaso-occlusive devices include, but not limited to, metal and/or polymeric devices. Suitable metals and metal alloys include the Platinum Group metals, especially platinum, rhodium, palladium, rhenium, as well as tungsten, gold, silver, tantalum, and alloys of these metals. The core element may also comprise of any of a wide variety of stainless steels. Very desirable materials of construction, from a mechanical point of view, are materials that maintain their shape despite being subjected to high stress including but not limited to “super-elastic alloys” such as nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum). Particularly preferred are the alloys described in U.S. Pat. Nos. 3,174,851; 3,351,463; and 3,753,700. Especially preferred is the titanium/nickel alloy known as “nitinol.”

The vaso-occlusive devices may be of any structure, for example, vaso-occlusive devices of tubular structures, for examples, braids, coils, combination braid and coils and the like. Thus, although depicted in the Figures described below as a coil, the vaso-occlusive device may be of a variety of shapes or configuration includes, but not limited to, braids, knits, woven structures, tubes (e.g., perforated or slotted tubes), cables, injection-molded devices and the like. See, e.g., U.S. Pat. No. 6,533,801 and International Patent Publication WO 02/096273. The vaso-occlusive device may change shape upon deployment, for example change from a constrained linear form to a relaxed, three-dimensional (secondary) configuration. See, also, U.S. Pat. No. 6,280,457. In a preferred embodiment, the core element comprises a metal wire wound into a primary helical shape.

The devices are often introduced into a selected site using the procedure outlined below. This procedure may be used in treating a variety of maladies. For instance in the treatment of an aneurysm, the aneurysm itself will be filled (partially or fully) with electrolytically detachable devices.

Conventional catheter insertion and navigational techniques involving guidewires or flow-directed devices may be used to access the site with a catheter as described herein. For use in peripheral or neural surgeries, the catheter will normally be about 100-200 cm in length, more normally 130-180 cm in length. The diameter of the delivery mechanism is usually in the range of 0.25 to about 0.90 mm. Briefly, occlusive devices (and/or additional components) are typically loaded into a carrier for introduction into the delivery catheter and introduced to the chosen site.

A selected site is reached through the vascular system using a collection of specifically chosen catheters and/or guide wires inserted using a vascular access sheath as described herein. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a sheath as described herein is introduced through an entry site in the vasculature. Typically, this would be through a femoral artery in the groin. Other entry sites sometimes chosen are found in the neck and are in general well known by physicians who practice this type of medicine. Once the sheath is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared and/or flushed, for example, with an electrolyte solution.

Once the selected site has been reached, the electrolytically detachable device is extruded and released in the desired position of the selected site.

Modifications of the procedure and catheters described above, and the methods of using them in keeping with the present disclosure will be apparent to those having skill in this mechanical and surgical art. These variations are intended to be within the scope of the claims that follow.

Modifications of the procedures and assemblies described above, and the methods of using them in keeping with this disclosure will be apparent to those having skill in this mechanical and surgical art. These variations are intended to be within the scope of the claims that follow.

What is claimed is:

1. A vascular access sheath comprising:
a tubular element having an inner and outer surface; and
an electrically conductive element on the inner and/or outer surface of the tubular element.

2. The vascular access sheath of claim 1, wherein the electrically conductive element is on the outer surface of the tubular element.

3. The vascular access sheath of claim 1, wherein the electrically conductive element comprises a layer of conductive material that at least partially covers the tubular element.

4. The vascular access sheath of claim 1, wherein the conductive layer comprises a metal.

5. The vascular access sheath of claim 4, wherein the metal is selected from the group of metals or alloys comprising at least one of gold, platinum, silver, tantalum, palladium, tungsten, titanium, iron, cobalt, chromium, nickel, aluminum, copper.

6. The vascular access sheath of claim 1, wherein the conductive layer comprises titanium-nitride, graphite.

7. The vascular access sheath of claim 1, wherein the conductive layer comprises a polymer.

8. The vascular access sheath of claim 1, wherein the electrically conductive element comprises one or more conductive elements selected from the group consisting of coatings, coils, braids, bands and wires.

9. The vascular access sheath of claim 1, further comprising a hydrophilic coating.

10. An assembly comprising:
a vascular access sheath according to claim 1;
a delivery catheter; and
an electrolytically detachable implantable device.

11. The assembly of claim 10, wherein the electrolytically detachable implantable device comprises a vaso-occlusive device.

12. The assembly of claim 11, wherein the vaso-occlusive device comprises a vaso-occlusive coil.

13. A method of delivering or receiving an electrical signal from a patient body, the method comprising:
introducing, into the vasculature of a subject, a vasculature access sheath according to claim 1; and
attaching the sheath to an electrical circuit.

14. The method of claim 13, where in the electrical circuit comprises an electrolytically detachable implant.

15. The method of claim 14, wherein the electrolytically detachable implant is deployed using a catheter that extends into the vasculature through the vasculature access sheath.

16. The method of claim 14, wherein the electrolytically detachable implantable device comprises a vaso-occlusive device.

17. The assembly of claim 16, wherein the vaso-occlusive device comprises a vaso-occlusive coil.

18. A method of occluding a body, cavity comprising;
introducing, into the vasculature of a subject, a vasculature access sheath according to claim 1; and
deploying an electrolytically detachable vaso-occlusive device into the vasculature of the patient through the vascular access sheath.

19. The method of claim 18, wherein the body cavity is an aneurysm.
